(19) United States
(12) Patent Application Publication
(10) Pub. No.: US 2026/0335492 A1
(43) Pub. Date: Oct. 29, 2026

(54) AROMA OIL DIFFUSER

(71) Applicant: Jong Hyeon LEE, Seoul (KR)

(72) Inventor: Jong Hyeon LEE, Seoul (KR)

(21) Appl. No.: 19/365,198

(22) Filed: Oct. 22, 2025

(30) Foreign Application Priority Data
Oct. 23, 2024 (KR) 10-2024-0145479

(51) Int. Cl. A61L 9/14

(52) U.S. Cl. CPC A61L 9/14; A61L 2209/135

(57) ABSTRACT

The present invention relates to an aroma oil diffuser including a lower body portion, a column portion coupled to one side of an upper surface of the lower body portion, and an upper body portion of which one side of a lower surface is coupled to the column portion, wherein the upper body portion has an aroma oil container coupled to the other side of the lower surface thereof and has a spray hole through which a vaporized aroma oil is sprayed formed in the other side of an upper surface. According to the present invention, aroma oil containers of various sizes and shapes can be applied, and since an aroma oil container is directly fastened to a vaporizing portion, there is an advantageous effect that an aroma oil can be more easily pumped and then vaporized.

### CROSS-REFERENCE TO RELATED APPLICATION

This application claims priority to and the benefit of Korean Patent Application No. 10-2024-0145479, filed on Oct. 23, 2024, the disclosure of which is incorporated herein by reference in its entirety.

### BACKGROUND

### 1. Field of the Invention

The present invention relates to an aroma oil diffuser, and more particularly, to an aroma oil diffuser using a nebulizing technology.

### 2. Discussion of Related Art

Unlike typical aroma oil diffusers using ultrasonic waves or heat, an aroma oil diffuser using a nebulizing technology breaks an essential oil into fine particles in micro units by only using air pressure without water or heat based on Bernoulli's principle and disperses the fine particles in the air.

Such a nebulizing diffuser has the following main features.

First, since the nebulizing diffuser does not need water or heating and only uses a pure essential oil, purity of the oil can be maintained, and the natural fragrance and efficacy of the oil can be maximized.

In addition, the nebulizing diffuser breaks an essential oil into fine particles and sprays the fine particles in the air, and since the fine particles stay in the air for a long period of time and are able to rapidly and evenly spread in a wide space, a user can experience a strong fragrance and efficacy of the oil rapidly and for a long period of time.

Further, the nebulizing diffuser also has an advantage of being suitable for use in a quiet environment due to hardly generating noise during operation.

Meanwhile, Korean Patent Registration No. 10-2581216, which is related art, relates to a diffuser and discloses a structure in which an aroma container (30) is mounted on a lower portion of a body (10), and an aroma chemical held in the aroma container (30) is discharged to an outlet (711) of an upper surface of the body (10) through a flow path member (70) inside.

However, in the diffuser of the related art, since the aroma container (30) is mounted on the lower portion of the body (10), and the aroma container (30) supports the body (10), there is a problem that it is not possible to apply aroma containers (30) of various sizes and shapes in some cases such as when a diameter of the aroma container (30) is smaller than a diameter of the body (10).

Further, when the aroma chemical is conveyed from the aroma container (30) at the lower portion to the outlet (711) at the upper portion through the flow path member (70), due to the high viscosity characteristic of an essential oil, there is a disadvantage that an insufficient amount of aroma chemical is conveyed, and discharge of the aroma chemical is not properly performed.

### RELATED ART DOCUMENT

### Patent Document

(Patent Document 0001) Korean Patent Registration No. 10-2581216

### SUMMARY OF THE INVENTION

The present invention is directed to providing an aroma oil diffuser using a nebulizing technology that allows aroma containers of various sizes to be applied and allows an aroma oil to be easily pumped and then vaporized.

The objective of the present invention is achieved by an aroma oil diffuser including a lower body portion, a column portion coupled to one side of an upper surface of the lower body portion, and an upper body portion of which one side of a lower surface is coupled to the column portion, wherein the upper body portion has an aroma oil container coupled to the other side of the lower surface thereof and has a spray hole through which a vaporized aroma oil is sprayed formed in the other side of an upper surface.

In addition, the aroma oil may be vaporized using a nebulizing technology.

In addition, the aroma oil diffuser of the present invention may further include an air pump portion that is installed at one side of an inner portion of the upper body portion and generates a high-pressure air, a vaporizing portion that is installed at the other side of the inner portion of the upper body portion, receives the high-pressure air from the air pump portion, and vaporizes the aroma oil, and a tube that has one side connected to the air pump portion and the other side connected to the vaporizing portion to convey the high-pressure air in a horizontal direction.

In addition, the vaporizing portion may include a main body portion that receives the high-pressure air from the air pump portion and vaporizes the aroma oil and a fastening portion that is formed at a lower portion of the main body portion and to which the aroma oil container is fastened by a screw coupling method, and the fastening portion may be formed of a metal material.

In addition, the upper body portion may be provided to be rotatable at a predetermined angle with the column portion as an axis of rotation.

### BRIEF DESCRIPTION OF THE DRAWINGS

The above and other objects, features, and advantages of the present invention will become more apparent to those of ordinary skill in the art by describing exemplary embodiments thereof in detail with reference to the accompanying drawings, in which:

FIG. 1 is an overall view of an aroma oil diffuser according to one embodiment of the present invention;

FIG. 2 is a front view of the aroma oil diffuser according to one embodiment of the present invention;

FIG. 3 is a side view of the aroma oil diffuser according to one embodiment of the present invention;

FIG. 4 illustrates a state in which an upper body portion of the aroma oil diffuser according to one embodiment of the present invention is rotated;

FIG. 5 is a side cross-sectional view of the aroma oil diffuser according to one embodiment of the present invention;

FIG. 6 is a block diagram showing a connection relationship among components of the aroma oil diffuser according to one embodiment of the present invention;

FIG. 7 illustrates a state in which different adapters are mounted on a fastening portion of the aroma oil diffuser according to one embodiment of the present invention;

FIG. 8 illustrates an adapter storage space provided in a lower body portion of the aroma oil diffuser according to one embodiment of the present invention; and

FIG. 9 illustrates an auxiliary body portion coupled to the lower body portion of the aroma oil diffuser according to one embodiment of the present invention.

### DETAILED DESCRIPTION OF EXEMPLARY EMBODIMENTS

Hereinafter, some embodiments of the present invention will be described in detail with reference to the exemplary drawings. In assigning reference numerals to components of each drawing, it should be noted that like components are denoted by like reference numerals wherever possible even when the components are shown in different drawings.

In addition, in describing embodiments of the present invention, when detailed description of a related known configuration or function is determined as hindering understanding of the embodiments of the present invention, the detailed description thereof will be omitted.

In addition, in describing the components of the embodiments of the present invention, terms such as “first,” “second,” “A,” “B,” “(a),” and “(b)” may be used. Such terms are only for distinguishing one component from another component, and the essence, sequence, order, or the like of the corresponding component is not limited by the terms.

In addition, in describing the embodiments of the present invention, each “part,” “module,” or “step” may be implemented using a processor and a memory. The term “processor” should be interpreted in a wide sense to include a general-purpose processor, a central processing unit (CPU), a microprocessor, a digital signal processor (DSP), a controller, a microcontroller, a state machine, and the like. In some environments, “processor” may also refer to an application-specific integrated circuit (ASIC), a programmable logic device (PLD), a field programmable gate array (FPGA), or the like. For example, the term “processor” may also refer to a combination of processing devices such as a combination of a DSP and a microprocessor, a combination of a plurality of microprocessors, a combination of one or more microprocessors combined with a DSP core, or a combination of other arbitrary configurations.

In addition, the term “memory” should be interpreted in a wide sense to include an arbitrary electronic component that can store electronic information. The term “memory” may also refer to various types of processor-readable media such as a random access memory (RAM), a read-only memory (ROM), a nonvolatile random access memory (NVRAM), a programmable read-only memory (PROM), an erasable-programmable read-only memory (EPROM), an electrically erasable PROM (EEPROM), a flash memory, a magnetic or optical data storage device, and registers. When a processor is able to read information from a memory or record information in the memory, the memory is referred to as being in an electronic communication state with the processor, and each “part,” “module,” or “step” may be implemented through a program or an application based on a processor and a memory that are in an electronic communication state with each other.

An aroma oil diffuser 100 according to one embodiment of the present invention will now be described in detail with reference to the accompanying drawings.

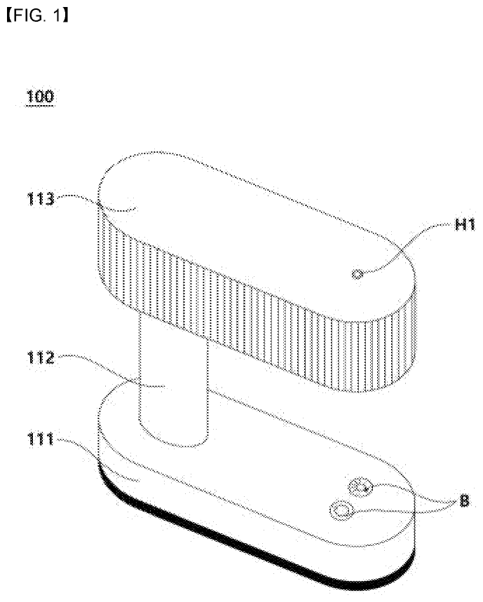

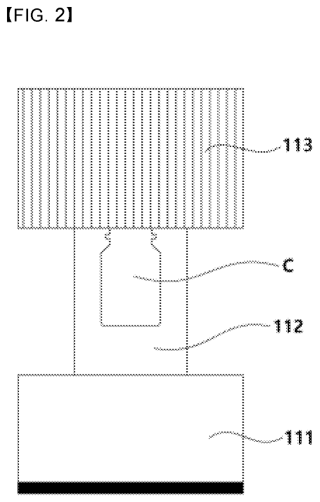

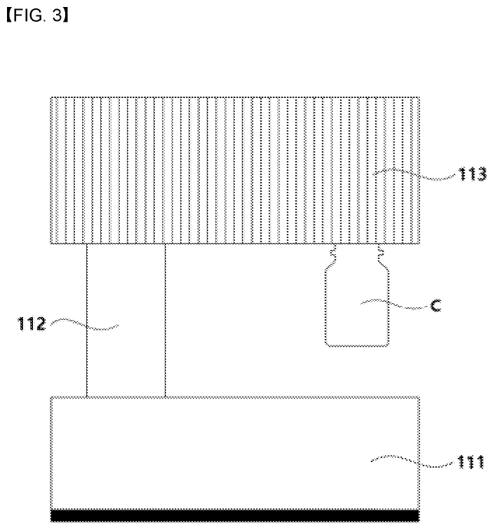

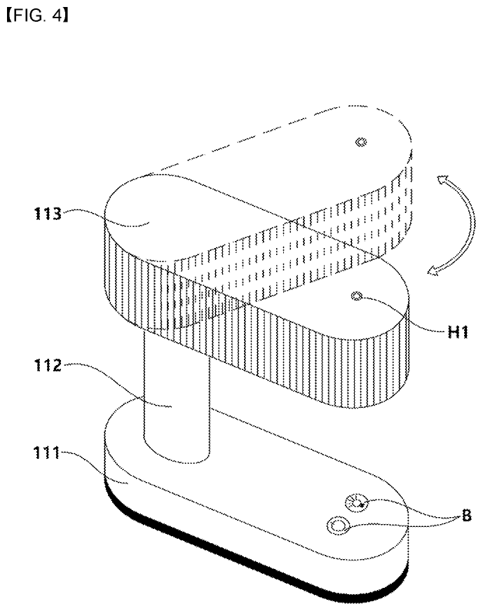

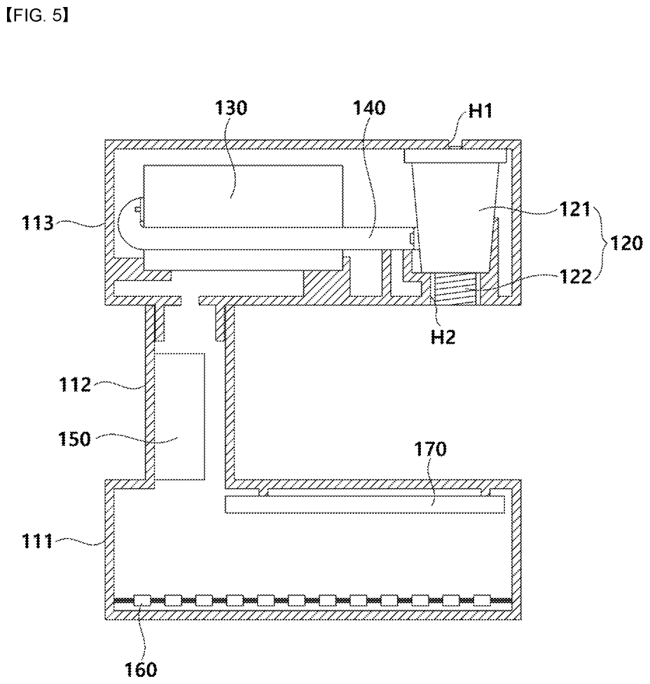

FIG. 1 is an overall view of an aroma oil diffuser according to one embodiment of the present invention, FIG. 2 is a front view of the aroma oil diffuser according to one embodiment of the present invention, FIG. 3 is a side view of the aroma oil diffuser according to one embodiment of the present invention, FIG. 4 illustrates a state in which an upper body portion of the aroma oil diffuser according to one embodiment of the present invention is rotated, FIG. 5 is a side cross-sectional view of the aroma oil diffuser according to one embodiment of the present invention, and FIG. 6 is a block diagram showing a connection relationship among components of the aroma oil diffuser according to one embodiment of the present invention.

As illustrated in FIGS. 1 to 6, the aroma oil diffuser 100 according to one embodiment of the present invention includes a lower body portion 111, a column portion 112, an upper body portion 113, a vaporizing portion 120, an air pump portion 130, a tube 140, a battery portion 150, a lighting portion 160, and a controller 170 First, the lower body portion 111, the column portion 112, and the upper body portion 113 constitute an exterior of the aroma oil diffuser 100 of the present invention and may be formed of a synthetic resin material, but the present invention is not limited thereto.

Among these components, the lower body portion 111 supports the column portion 112 and the upper body portion 113 which will be described below, the column portion 112 is coupled to an edge of one side of an upper surface of the lower body portion 111, a plurality of button portions B for controlling an operation of the aroma oil diffuser 100 of the present invention are provided on the other side of the upper surface of the lower body portion 111, and one portion of a lower portion of the lower body portion 111 is formed of a transparent material to allow transmission of light emitted from the lighting portion 160 installed therein.

Here, the button portions B may include a power button, an intensity control button, and/or a lighting button and may operate by touch, but the present invention is not limited thereto.

The column portion 112 is coupled to the edge of the one side of the upper surface of the lower body portion 111, supports the upper body portion 113 which will be described below, and is formed to extend in a height direction. The column portion 112 may have a hollow cylindrical shape, but the present invention is not limited thereto.

The upper body portion 113 is supported by the column portion 112 by an edge of one side of a lower surface of the upper body portion 113 being coupled to the column portion 112, a fastening hole H2 to which an aroma oil container C is coupled is formed in the other side of the lower surface of the upper body portion 113, and a spray hole H1 through which a vaporized aroma oil is sprayed is formed in the other rside of an upper surface of the upper body portion 113.

That is, in the present invention, since a sufficient space can be formed between the upper body portion 113 and the lower body portion 111 by the column portion 112 being coupled to one side edge of each of the upper body portion 113 and the lower body portion 111, aroma oil containers C of various sizes and shapes can be easily applied to the aroma oil diffuser 100.

In addition, as illustrated in FIG. 4, in the present invention, the upper body portion 113 may be provided to be rotatable at a predetermined angle with the column portion 112 as an axis of rotation. That is, the upper body portion 113 may be rotated 90° when a user applies an external force in one direction after gripping the upper body portion 113, and according to this, there is an effect that the user can more easily couple the aroma oil container C to the vaporizing portion 120 provided in the upper body portion 113 without interference of the lower body portion 111.

Meanwhile, although not illustrated in the drawings, a motor that applies a rotational force to rotate the upper body portion 113 in a fully automatic manner may be provided inside the column portion 112. Specifically, a pinion gear may be provided on an end of the motor, a ring gear may be provided inside the upper body portion 113, and the upper body portion 113 may be rotated in a fully automatic manner by the pinion gear and the ring gear being engaged with each other. At this time, the button portions B may additionally include a button for controlling a rotating operation of the upper body portion 113.

The vaporizing portion 120 pumps up an aroma oil from the aroma oil container C and then vaporizes the aroma oil using a nebulizing technology. The vaporizing portion 120 is installed at the other side of an inner portion of the upper body portion 113, has an upper portion installed to communicate with the spray hole H1 of the upper body portion 113 and a lower portion installed to communicate with the fastening hole H2 of the upper body portion 113, and is controlled by the controller 170 which will be described below.

Here, nebulizing is a method in which high-pressure air is used to convert a liquid, mostly, an aroma oil or an essential oil, into fine particles and diffuse the fine particles in the air, and as the high-pressure air passes through the oil, the oil breaks into particles at a molecular level, and a high-concentration fragrance is diffused. Since nebulizing can be performed by only using an air pressure without using heat or water, and thus pure oil particles can be released, a strong fragrance can be provided while purity of the fragrance is maintained.

More specifically, the vaporizing portion 120 includes a main body portion 121 and a fastening portion 122.

The main body portion 121 receives high-pressure air from the air pump portion 130 and vaporizes the aroma oil, and a spray port through which the vaporized aroma oil is sprayed is formed in an upper portion of the main body portion 121 and communicates with the spray hole H of the upper body portion 113.

The fastening portion 122 is formed at a lower portion of the main body portion 121, allows the aroma oil container C to be fastened thereto by a screw coupling method, and communicates with the fastening hole H2 of the upper body portion 113. The fastening portion 122 may be formed of a metal material such as aluminum and stainless steel, but the present invention is not limited thereto.

That is, in the present invention, since the aroma oil container C can be directly fastened to the vaporizing portion 120 and thus a conveying distance is short, there is an effect that a sufficient amount of aroma oil can be conveyed to the vaporizing portion 120 even in the case of an aroma oil having high viscosity.

In addition, according to the fastening portion 122 described above, since the portion to which the aroma oil container C is coupled is formed of a metal material, more firm coupling is possible, and since the fastening portion 122 is not easily damaged even with repeated fastening, there is an effect that durability of the aroma oil diffuser 100 can be significantly improved.

Meanwhile, in the present invention, to improve durability of the vaporizing portion 120, not only the fastening portion 122 but also the main body portion 121 may be formed of a metal material.

The air pump portion 130 generates high-pressure air and provides the high-pressure air to the vaporizing portion 120 to allow the vaporizing portion 120 to vaporize the aroma oil using the high-pressure air. The air pump portion 130 is installed at one side of an inner portion of the upper body portion 113 and is controlled by the controller 170 which will be described below.

The tube 140 has a flow path formed therein to convey the high-pressure air generated in the air pump portion 130 to the vaporizing portion 120 and has one end connected to the air pump portion 130 and the other end connected to the vaporizing portion 120.

Meanwhile, generally, in a conventional diffuser, the vaporizing portion 120 is disposed at an upper portion, the air pump portion 130 is disposed at a lower portion, and the high-pressure air is provided upward while opposing gravity. On the other hand, in the present invention, both the vaporizing portion 120 and the air pump portion 130 are installed in the upper body portion 113 while the air pump portion 130 is disposed beside the vaporizing portion 120, and thus, the high-pressure air can be easily conveyed in the horizontal direction, and an effect of lowering power consumption is also expected.

The battery portion 150 supplies power for an operation of the aroma oil diffuser 100 of the present invention. The battery portion 150 is installed inside the column portion 112 or the lower body portion 111 and is controlled by the controller 170 which will be described below.

The lighting portion 160 emits light of a predetermined color and/or pattern to produce lighting, is provided to include a plurality of light emitting diode (LED) modules or the like, is installed inside the lower body portion 111 while installed in a circumferential direction of the lower portion of the lower body portion 111, and is controlled by the controller 170 which will be described below. At this time, one portion of the lower portion of the lower body portion 111 is formed of a transparent material to allow transmission of light emitted from the lighting portion 160.

The controller 170 controls an operation of the aroma oil diffuser 100 of the present invention, is electrically connected to the vaporizing portion 120, the air pump portion 130, the battery portion 150, and the lighting portion 160, and is installed inside the lower body portion 111.

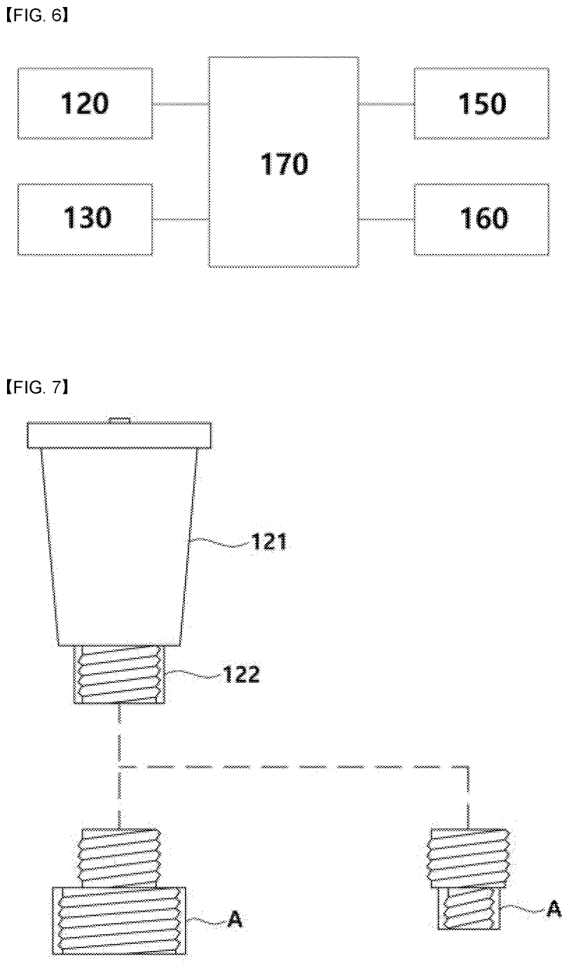

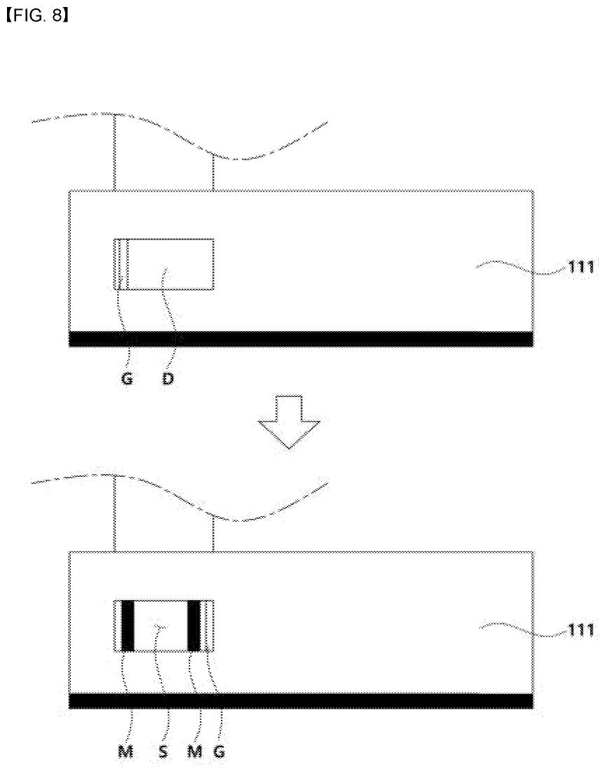

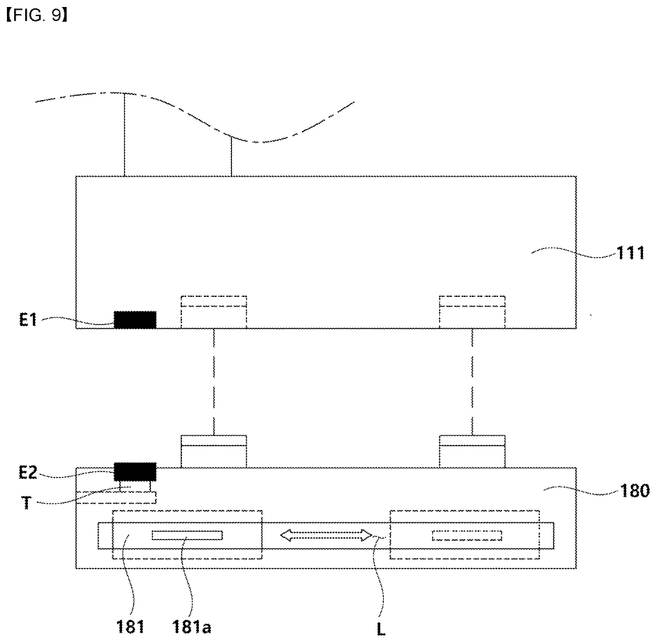

FIG. 7 illustrates a state in which different adapters are mounted on a fastening portion of the aroma oil diffuser according to one embodiment of the present invention, and FIG. 8 illustrates an adapter storage space provided in a lower body portion of the aroma oil diffuser according to one embodiment of the present invention.

Meanwhile, in the aroma oil diffuser 100 of the present invention, a sufficient space is formed between the upper body portion 113 and the lower body portion 111, and aroma oil containers C of various sizes and shapes can be applied, and accordingly, an outer diameter of an inlet portion may be different for each aroma oil container C.

Accordingly, as illustrated in FIG. 7, the aroma oil diffuser 100 of the present invention may further include a plurality of adapters A having different inner diameters to allow aroma oil containers C having inlet portions of different outer diameters to be fastened to the fastening portion 122.

In addition, as illustrated in FIG. 8, an adapter accommodation space S that can accommodate the plurality of adapters A mentioned above may be formed inside the lower body portion 111, and a pair of magnet portions M may be provided on both side surfaces of the adapter accommodation space S to fix the adapters A made of a metal material using a magnetic force. In addition, a door portion D that has a grip portion G provided thereon and is opened and closed in a sliding manner may be formed on a side surface of the lower body portion 111 to allow the above-mentioned adapter accommodation space S to be opened and closed in a sliding manner.

According to the above, the aroma oil containers C having inlet portions of different outer diameters can be fastened to the fastening portion 122 through the plurality of adapters A, and there is an effect that the plurality of adapters A can be easily stored inside the aroma oil diffuser 100.

FIG. 9 illustrates an auxiliary body portion coupled to the lower body portion of the aroma oil diffuser according to one embodiment of the present invention.

Meanwhile, the battery portion 150 of the aroma oil diffuser 100 of the present invention may be charged in a USB Type-C manner or the like, and the aroma oil diffuser 100 of the present invention may also operate in a wireless manner after the battery portion 150 is charged. At this time, during a wireless operation, a long-term operation may be difficult due to an insufficient capacity of the battery portion 150 in some cases.

In addition, aroma oil containers C of various sizes and shapes may be applied to the aroma oil diffuser 100 of the present invention, and when an aroma oil container C with a large volume or weight is fastened to the fastening portion 122, there is a disadvantage that the diffuser may fall down due to a change in center of mass.

Accordingly, as illustrated in FIG. 9, the aroma oil diffuser 100 of the present invention may further include an auxiliary body portion 180 that has an auxiliary battery portion 181 provided therein and is detachably coupled in a snap-fit manner to the lower portion of the lower body portion 111. At this time, a first electrode El electrically connected to the controller 170 may be formed on a lower surface of the lower body portion 111, a second electrode E2 electrically connected to the auxiliary battery portion 181 may be formed on an upper surface of the auxiliary body portion 180, and when the lower body portion 111 and the auxiliary body portion 180 are coupled, the first electrode E1 and the second electrode E2 may come into contact with each other and be electrically connected. At this time, an elastic body T is provided on a lower surface of the second electrode E2 to allow the second electrode E2 to more easily come into contact with the first electrode E1.

In addition, a slide groove L may be formed in one side surface or both side surfaces of the auxiliary body portion 180 in the longitudinal direction, and a blade portion 181a may be formed on one side surface or both side surfaces of the auxiliary battery portion 181 and may protrude to an outer portion of the slide groove. That is, in the present invention, a user is able to adjust the position of the auxiliary battery portion 181 along the slide groove L by applying an external force after gripping the blade portion 181a, and accordingly, center of mass of the aroma oil diffuser 100 of the present invention can be adjusted.

According to the present invention, aroma oil containers of various sizes and shapes can be applied, and since an aroma oil container is directly fastened to a vaporizing portion, there is an advantageous effect that an aroma oil can be more easily pumped and then vaporized.

In addition, according to the present invention, since a fastening portion to which an aroma oil container is coupled is formed of a metal material, more firm coupling is possible, and since the fastening portion is not easily damaged even with repeated fastening, there is an advantageous effect that durability of the aroma oil diffuser can be significantly improved.

Meanwhile, advantageous effects of the present invention are not limited to those mentioned above, and various other effects may be included within the scope self-evident to those of ordinary skill in the art from the content described above.

Even if all the components constituting the embodiments of the present invention have been described above as being combined into one or operating in combination, the present invention is not necessarily limited to these embodiments. That is, within the scope of the present invention, all of the components may be selectively combined and operated as one or more.

In addition, the terms “include,” “consist,” or “have” as described above mean that a corresponding component may be intrinsic, unless specifically stated otherwise, and it should be interpreted as including other components rather than excluding other components. All terms including technical or scientific terms have the same meanings as those commonly understood by those skilled in the art to which the present invention pertains, unless defined otherwise. Commonly used terms such as those defined in a dictionary should be construed as being consistent with the context of the relevant art, and are not to be construed in an idealized or overly formal sense unless expressly defined in the present invention.

Further, the above description is merely illustrative of the technical idea of the present invention, and those skilled in the art to which the present invention pertains may make various modifications and variations without departing from the essential characteristics of the present invention.

Therefore, the embodiments disclosed in the present invention are not intended to limit the technical spirit of the present invention, but to explain, and the scope of the technical spirit of the present invention is not limited by these embodiments. The scope of protection of the present invention should be construed from the following claims, and all technical ideas within the scope equivalent thereto should be construed as being included within the scope of rights of the present invention.

What is claimed is:

1. An aroma oil diffuser comprising:
a lower body portion;
a column portion coupled to one side of an upper surface of the lower body portion; and
an upper body portion of which one side of a lower surface is coupled to the column portion,
wherein the upper body portion has an aroma oil container coupled to the other side of the lower surface thereof and has a spray hole through which a vaporized aroma oil is sprayed formed in the other side of an upper surface.

2. The aroma oil diffuser of claim 1, wherein the aroma oil is vaporized using a nebulizing technology.

3. The aroma oil diffuser of claim 2, further comprising:
an air pump portion that is installed at one side of an inner portion of the upper body portion and generates a high-pressure air;
a vaporizing portion that is installed at the other side of the inner portion of the upper body portion, receives the high-pressure air from the air pump portion, and vaporizes the aroma oil; and
a tube that has one side connected to the air pump portion and the other side connected to the vaporizing portion to convey the high-pressure air in a horizontal direction.

4. The aroma oil diffuser of claim 3, wherein the vaporizing portion includes:
a main body portion that receives the high-pressure air from the air pump portion and vaporizes the aroma oil; and
a fastening portion that is formed at a lower portion of the main body portion and to which the aroma oil container is fastened by a screw coupling method, and
the fastening portion is formed of a metal material.

5. The aroma oil diffuser of claim 1, wherein the upper body portion is provided to be rotatable at a predetermined angle with the column portion as an axis of rotation.
